(19) United States
(12) Patent Application Publication
(10) Pub. No.: US 2019/0070174 A1
(43) Pub. Date: Mar. 7, 2019

(54) METHODS OF TREATING NEURODEGENERATIVE DISEASES

(71) Applicant: THE REGENTS OF THE UNIVERSITY OF MICHIGAN, Ann Arbor, MI (US)

(72) Inventors: Maria Do Carmo Pereira Da Costa, Ann Arbor, MI (US); Henry L. Paulson, Ann Arbor, MI (US)

(21) Appl. No.: 16/083,750

(22) PCT Filed: Mar. 10, 2017
(86) PCT No.: PCT/US17/21858

§ 371 Date: Sep. 10, 2018

Related U.S. Application Data
(60) Provisional application No. 62/306,218, filed on Mar. 10, 2016.

(51) Int. Cl. A61K 31/496; A61P 25/28

(52) U.S. Cl. CPC A61K 31/496; A61P 25/28

(57) ABSTRACT

Methods of using the antipsychotic drug aripiprazole in treating a neurodegenerative disease such as Machado-Joseph disease/Spinocerebellar ataxia type 3 (MJD/SCA3) are disclosed. The methods comprise administering aripiprazole in an amount effect to decrease protein aggregates in the central nervous system and intracellular forms of pathogenic proteins such as mutant ataxin-3.

### CROSS-REFERENCE TO RELATED APPLICATION

This application claims the benefit of priority under 35 U.S.C. § 119(e) of U.S. Provisional Patent Application No. 62/306,218 filed Mar. 10, 2016, which is incorporated herein by reference.

### STATEMENT OF GOVERNMENT INTEREST

This invention was made with government support under NS038712 awarded by the National Institutes of Health. The government has certain rights in the invention.

### FIELD OF THE INVENTION

The present disclosure relates to methods of treating neurodegenerative diseases such as Machado-Joseph disease (MJD), also known as Spinocerebellar ataxia type 3 disease (SCA3).

### BACKGROUND

Machado-Joseph disease (MJD), also known as Spinocerebellar ataxia type 3 (SCA3), is a fatal neurodegenerative disease. The most prevalent dominant hereditary ataxia, MJD/SCA3 is characterized by progressive ataxia, ophthalmoplegia and pyramidal signs often accompanied by extrapyramidal signs (Paulson, H. L. Semin Neurol 27, 133-142 (2007)). These clinical features reflect neuronal degeneration and pathological changes in the cerebellum, brainstem, substantia nigra, thalamus, basal ganglia, and spinal cord. MJD/SCA3 is one of nine known polyglutamine (polyQ) diseases caused by expanded CAG repeats that encode abnormally long polyQ tracts in the disease proteins. Other polyQ diseases include Dentatorubropallidoluysian atrophy (DRPLA), Huntington's disease (HD), Spinal and bulbar muscular atrophy (SMBA) and Spinocerebellar ataxia types 1, 2, 6, 7, and 17. In MJD/SCA3, the polyQ expansion occurs near the carboxyl-terminus of ataxin-3 (ATXN3), a deubiquitinase encoded by the ATXN3 gene. While normal ATXN3 alleles contain 12 to 44 CAG repeats, disease alleles are expanded to about 60 to 87 triplets (Lima et al. Hum Hered 60, 156-163 (2005)). The polyQ expansion in ATXN3 increases its propensity to aggregate, leading to the formation of intracellular aggregates (Costa Mdo, C. & Paulson, H. L. Prog Neurobiol 97, 239-257 (2012)). These aggregates are found in the nuclei of neurons as large inclusions (Paulson et al. Neuron 19, 333-344 (1997)), but also occur in the cytoplasm and neuritis, usually as smaller puncta (Hayashi et al. Psychiatry Clin Neurosci 57, 205-213 (2003)). ATXN3 is known to regulate the stability of proteins involved in diverse pathways (Todi, S. V. & Paulson, H. L. Trends Neurosci 34, 370-382, (2011)), but ATXN3 carrying an expanded polyQ tract becomes neurotoxic and triggers several pathogenic cascades (Matos et al. Prog Neurobiol 95, 26-48 (2011)).

Molecular chaperones, the proteasome, and macroautophagy are components of cellular protein quality control (PQC) known to regulate mutant ATXN3 and other proteins and/or promote their degradation (Teixeira-Castro et al. Hum Mol Genet 20, 2996-3009 (2011)). Reducing the abundance of mutant ATXN3 or its oligomers is a compelling therapeutic approach because these species represent upstream targets in the pathogenic cascade. Moreover, the fact that mice lacking ATXN3 appear normal (Schmitt et al. Biochem Biophys Res Commun 362, 734-739 (2007)) suggests that strategies to reduce total levels of ATXN3 (i.e., normal and mutant) should not result in adverse consequences due to loss of ATXN3 function. Although gene-silencing strategies that effectively reduce levels of mutant ATXN3 in MJD/SCA3 mouse models have been developed (do Carmo Costa, M. et al. Mol Ther 21, 1898-1908 (2013); Nobrega, C. et al. PLoS One 8, e52396 (2013); Rodriguez-Lebron, E. et al. Mol Ther 21, 1909-1918 (2013)), achieving broad brain delivery of silencing agents for optimal efficacy in MJD/SCA3 patients remains a challenge. No disease-modifying or preventative therapy exists for MJD/SCA3 and other neurodegenerative diseases, so there remains a need for therapeutic options for MJD/SCA3 and other neurological diseases associated with accumulation of toxic proteins in the central nervous system.

### SUMMARY

The present disclosure is directed to methods of treating a neurodegenerative disease such as MJD/SCA3 in a subject in need thereof comprising administering a therapeutically effective amount of aripiprazole. In one aspect, a method of treating a neurodegenerative disease in a subject in need thereof comprises administering aripiprazole in an amount effective to reduce protein aggregates in the central nervous system of the subject. The methods of the present disclosure may be used to treat a neurodegenerative disease, including neurodegenerative proteinopathies such as polyglutamine (polyQ) diseases, optionally selected from the group consisting of Alzheimer's disease, Parkinson's disease, amyotrophic lateral sclerosis (ALS), frontotemporal dementia, spinocerebellar ataxia (SCA) type 1, SCA type 2, SCA type 6, SCA type 7, SCA type 17, Machado-Joseph disease/SCA type 3 (MJD/SCA3), Huntington's disease, dentatorubral pallidoluysian atrophy (DRPLA), spinal and bulbar muscular atrophy, and X-linked 1 (SBMA). In one aspect, the methods comprise administering aripiprazole in an amount effective to reduce protein aggregates in an area of the central nervous system of the subject selected from the brainstem, cerebellum, spinal cord, forebrain, and combinations thereof.

In another aspect, a method of treating a polyQ disease is provided comprising administering a therapeutically effective amount of aripiprazole. In one aspect, a method of the disclosure comprises administering aripiprazole in an amount effective to decrease levels of a mutant protein having an expanded polyglutamine tract. In another aspect, a method of treating MJD/SCA3 comprises administering aripiprazole in an amount effective to decrease ataxin-3 (ATXN3) levels, protein aggregates comprising ATXN3, and/or high molecular weight ATXN3 species. In one aspect, a method of reducing intracellular ATXN3 levels comprises contacting a cell with an effective amount of aripiprazole, for example, a neuron or glial cell.

The foregoing summary is not intended to define every aspect of the invention, and other features and advantages of the present disclosure will become apparent from the following detailed description, including the drawings. The present disclosure is intended to be related as a unified document, and it should be understood that all combinations of features described herein are contemplated, even if the combination of features are not found together in the same sentence, paragraph, or section of this disclosure. In addition, the disclosure includes, as an additional aspect, all embodiments of the invention narrower in scope in any way than the variations specifically mentioned above. With respect to aspects of the disclosure described or claimed with “a” or “an,” it should be understood that these terms mean “one or more” unless context unambiguously requires a more restricted meaning. With respect to elements described as one or more within a set, it should be understood that all combinations within the set are contemplated. If aspects of the disclosure are described as “comprising” a feature, embodiments also are contemplated “consisting of” or “consisting essentially of” the feature. Additional features and variations of the disclosure will be apparent to those skilled in the art from the entirety of this application, and all such features are intended as aspects of the disclosure.

### BRIEF DESCRIPTION OF THE DRAWINGS

FIG. 1A to FIG. 1I depict luminescence and viability dose-response screens identifying actives for follow-up studies. FIG. 1A to FIG. 1I represent cell viability (dark bars) and luminescence inhibition (light bars) relative to vehicle in dose-response screens for nine compounds that passed triage criteria (FIG. 1A: artemether, FIG. 1B: monensin sodium, FIG. 1C: salinomycin sodium, FIG. 1D: tranilast, FIG. 1E: AM251, FIG. 1F: mifepristone, FIG. 1G: aripiprazole, FIG. 1H: clotrimazole, and FIG. 1I: cefamandole sodium). Percentages of viability and luminescence inhibition relative to vehicle-treated cells are represented as the mean of duplicates ±SEM. The IC50 of luminescence inhibition for each compound is also indicated.

FIG. 2A and FIG. 2B depict the effects of five small molecules (sodium salinomycin (Na Sal), AM251, aripiprazole (Arip), clotrimazole (Clotrim), and mifepristone (Mifep)) on levels of expanded ATXN3 in confirmation screens using 293.ATXN3Q81.Luc cells. FIG. 2A shows quantification of ATXN3Q81Luc, and FIG. 2B shows quantification of endogenous ATXN3 (endATXN3). Bars represent the mean percentage of each protein relative to vehicle-treated cells and normalized to α-Tubulin (±SEM) in three independent experiments. Comparisons between cells treated with a specific compound concentration and cells treated with vehicle were performed using Student's t-test with statistical significance, as indicated: *P<0.05 and **P<0.01.

FIG. 3A and FIG. 3B depict the effects of sodium salinomycin, AM251, and aripiprazole on human mutant ATXN3 levels in organotypic brain slice cultures from YACMJD84.2 transgenic mice (Q84). FIG. 3A depicts levels of human mutant ATXN3, and FIG. 3B depicts levels of mouse Atxn3. Bars represent the mean percentage of protein relative to levels in vehicle-treated slices and normalized to α-Tubulin (±SEM) for three independent experiments using different mice. Comparison between slices treated with a specific compound/concentration and slices treated with vehicle was performed using Student's t-test with statistical significance, as indicated: *P<0.05 and **P<0.01.

FIG. 4A to FIG. 4C depict the effect of aripiprazole on the longevity of flies expressing mutant ATXN3 and on high molecular weight (HMW) ATXN3 species. FIG. 4A depicts Kaplan-Meier survival curves showing that MJD/SCA3 flies (n=318) have a markedly shortened lifespan compared to flies containing empty vector control (CTRL) (n=284). FIG. 4B depicts the survival of MJD/SCA3 flies that upon eclosion, were placed in instant formula food containing either the vehicle (1:1 DMSO:Tween-80) or aripiprazole (50 μM). Aripiprazole-treated flies (Arip) (n=242) showed a significant increase in mean survival compared to vehicle-treated flies. Kaplan-Meier survival curves were compared using the Log-Rank Mantel-Cox test. **represents a P<0.01 and ***represents a P<0.001. FIG. 4C depicts the relative amount of HMW ATXN3 species to total ATXN3 measuring using an immunoblot in flies treated with aripiprazole (light bars) and vehicle (dark bars).

FIG. 5A to FIG. 5F depict the effects of subchronic treatment of Q84 mice with aripiprazole on soluble aggregates of ATXN3 in the brainstem/midbrain. FIG. 5A depicts anti-ATXN3 immunoblotting of soluble protein extracts of brainstem revealing decreased HMW ATXN3 species in aripiprazole-treated mice. FIG. 5B depicts ATXN3 species showing that aripiprazole reduced HMW ATXN3 species to 44% of levels found in vehicle-treated mice. Bars represent the average percentage of protein species relative to vehicle-treated mice, corrected for α-Tubulin (±SEM). Comparison between groups was made using Student's t-test and statistical significance is indicated as *for P<0.05. FIG. 5C depicts aripiprazole and vehicle-treated mice showing similar levels of human ATXN3 and mouse Atxn3 transcripts in brainstem. Values were normalized for Gapdh expression and referenced to the average of vehicle-treated mice of the correspondent gender. Bars represent the average of transcript fold change per mouse group (N=9)±SEM. FIG. 5D depicts insoluble ATXN3 in the brainstem/midbrain of aripiprazole and vehicle-treated mice. Bars represent the average of insoluble ATXN3 relative to vehicle-treated mice (±SEM). FIG. 5E depicts the number of ATXN3-positive puncta in ventral pontine nuclei in aripiprazole and vehicle-treated mice. Bars represent the average of puncta (±SEM). FIG. 5F depicts nuclear ATXN3 fluorescence in pontine neurons in aripiprazole and vehicle-treated mice. Bars correspond to the average corrected total cell fluorescence (CTCF) of ATXN3 (±SEM).

FIG. 6 depicts a Thioflavin T (ThT) fluorescence assay showing that ATXN3Q55 fibril formation was not affected by aripiprazole. Curves of recombinant ATXN3Q55 (10 μM) incubated with 40 times molar excess of aripiprazole (400 μM) (light line) or vehicle (dark line) were normalized by fluorescence values for the blank control (buffer). Each point on the ThT fluorescence assays corresponds to the average of three replicates in two independent experiments.

FIG. 7A to FIG. 7G depict dysregulation of key proteostasis components in Q84 mice from Western blot analysis of total soluble protein lysates from brainstems of 12-week old Q84 mice (n=6) and wild type littermate controls (wt) (n=6). FIG. 7A depicts Hsp70 levels, FIG. 7B depicts Hsp40 levels, FIG. 7C depicts Hsp90a levels, FIG. 7D depicts Hsp90β levels, FIG. 7E depicts Hsf1 levels, FIG. 7F depicts Rad23a levels, and FIG. 7G depicts Rad23b levels. Bars represent the average percentage of protein relative to vehicle-treated mice (±SEM). Comparison between groups was made using Student's t-test and statistical significance is indicated as **for P<0.01, and ***for P<0.001.

FIG. 8A to FIG. 8I depict the effect of aripiprazole on components of the protein quality control machinery in brains of treated Q84 mice. FIG. 8A depicts Hsp70 levels, FIG. 8B depicts Hsp40 levels, FIG. 8C depicts Hsp90a levels, FIG. 8D depicts Hsp90β levels, FIG. 8E depicts Hsf1 levels, FIG. 8F depicts Rad23a levels, FIG. 8G depicts Rad23b levels, FIG. 8H depicts high molecular weight (HMW) Ub levels, and FIG. 8I depicts total Ub levels. Bars represent the average percentage of protein relative to vehicle-treated mice (±SEM). Comparison between groups was made using Student's t-test and statistical significance is indicated as * for P<0.05, ** P<0.01, and *** P<0.001.

### DETAILED DESCRIPTION

The present disclosure provides methods of treating a neurodegenerative disease such as MJD/SCA3 in a subject in need thereof comprising administering a therapeutically effective amount of aripiprazole. Aripiprazole was identified using a cell-based screen as capable of reducing levels of mutant ATXN3. Aripiprazole increased longevity in a Drosophila model of MJD/SCA3 and effectively reduced aggregated ATXN3 species in the flies, as well as in the brains of MJD/SCA3 transgenic mice.

Without intending to be bound by theory, treatment with aripiprazole could affect the abundance of molecular chaperones in the brains, thereby decreasing misfolded, aggregated ATXN3 species. The ability of aripiprazole to help with clearing toxic ATXN3 from the brains of MJD/SCA3 subjects would also be beneficial for clearing toxic proteins expressed in other neurodegenerative proteinopathies, such as other polyglutamine (polyQ) diseases.

The following definitions may be useful in aiding the skilled practitioner in understanding the disclosure. Unless otherwise defined herein, scientific and technical terms used in the present disclosure shall have the meanings that are commonly understood by those of ordinary skill in the art.

The term “aripiprazole” refers to the compound having the molecular formula C₂₃H₂₇Cl₂₃O₂ and pharmaceutical compositions comprising the same, for example, as described in U.S. Pat. Nos. 7,053,092; 8,017,615, 8,759,350; and 9,125,939; incorporated herein by reference. The term encompasses the compound and formulation marketed as ABILIFY® in the United States, generic versions thereof, and deuterated forms, for example, as described in U.S. Patent Publication No. 2008/0299216, incorporated herein by reference.

The term “neurodegenerative disease” refers to a condition associated with a progressive loss of structure and/or function of neurons. One type of neurodegenerative diseases is a “neurodegenerative proteinopathy” which refers to a neurodegenerative disease associated with the accumulation of mutant and toxic proteins in the central nervous system, such as a polyglutamine (polyQ) disease. Examples of neurodegenerative diseases include, but are not limited to, Alzheimer's disease, Parkinson's disease, amyotrophic lateral sclerosis (ALS), frontotemporal dementia, spinocerebellar ataxia (SCA) type 1, SCA type 2, SCA type 6, SCA type 7, SCA type 17, Machado-Joseph disease/SCA type 3 (MJD/SCA3), Huntington's disease, dentatorubral pallidoluysian atrophy (DRPLA), and spinal and bulbar muscular atrophy, X-linked 1 (SBMA).

The terms “therapeutically effective amount” and “effective amount” depend on the condition of a subject and dosing regimen. The terms refer to an amount of aripiprazole effective to achieve a desired biological, e.g., clinical effect. A therapeutically effective amount varies with the nature of the disease being treated, the length of time that activity is desired, and the age and the condition of the subject. In one aspect, a therapeutically effective amount of aripiprazole according to the disclosure is an amount effective to decrease intracellular levels of a mutant protein, decrease protein aggregates and high molecular weight species, promote degradation of a mutant protein and/or increase longevity.

The term “high molecular weight species” refers to a mutant protein or aggregate of mutant and/or wild-type proteins having a molecular weight that is at least two-fold greater than the molecular weight of the wild-type protein. For example, a high molecular weight species can have a molecular weight that is at least two-fold, at least three-fold, at least four-fold, at least five-fold, at least six-fold, at least seven-fold, at least eight-fold, at least nine-fold, at least ten-fold, or greater, than the molecular weight of the related wild-type protein.

In jurisdictions that forbid the patenting of methods that are practiced on the human body, the meaning of “administering” a composition to a human subject shall be restricted to prescribing a controlled substance that a human subject will self-administer by any technique (e.g., orally, inhalation, topical application, injection, insertion, etc.) and to the manufacture of a medicament for use in the methods described herein. The broadest reasonable interpretation that is consistent with laws or regulations defining patentable subject matter is intended. In jurisdictions that do not forbid the patenting of methods that are practiced on the human body, “administering” compositions includes both methods practiced on the human body and also the foregoing activities.

In one aspect, the disclosure provides a method of treating a neurodegenerative disease in a subject in need thereof comprising administering aripiprazole in an amount effective to reduce protein aggregates in the central nervous system of the subject. In one aspect, the subject has a neurodegenerative disease, optionally a neurodegenerative disease selected from Alzheimer's disease, Parkinson's disease, amyotrophic lateral sclerosis (ALS), frontotemporal dementia, SCA type 1, SCA type 2, SCA type 6, SCA type 7, SCA type 17, MJD/SCA3, Huntington's disease, DRPLA, and SBMA. In one aspect, the method comprises administering aripiprazole in an amount effective to decrease protein aggregates and/or high molecular weight species in an area of the central nervous system selected from the brainstem, cerebellum, spinal cord, forebrain, and combinations thereof.

In one aspect, the disclosure provides a method of treating a polyglutamine (polyQ) disease in a subject in need thereof comprising administering a therapeutically effective amount of aripiprazole, for example, polyQ disease is selected from SCA type 1, SCA type 2, SCA type 6, SCA type 7, SCA type 17, MJD/SCA3, Huntington's disease, DRPLA, and SBMA. In another aspect, aripiprazole is administered in an amount effective to decrease protein aggregates and/or high molecular weight species in the central nervous system of the subject, for example, protein aggregates and/or HMW species comprising a mutant protein having an expanded polyQ tract.

In one aspect, the disclosure provides a method of treating MJD/SCA3 in a subject in need thereof comprising administering a therapeutically effective amount of aripiprazole. In one aspect, the method comprises administering aripiprazole in an amount effective to reduce ataxin-3 (ATXN3) in the central nervous system, for example, in the brainstem, cerebellum, spinal cord, forebrain, and combinations thereof. In another aspect, the method comprises administering aripiprazole in an amount effective to decrease high molecular weight ATXN3 species and/or ATXN3 aggregates.

The disclosure also provides use of aripiprazole in the treatment of a neurodegenerative disease in a subject in need thereof, wherein aripiprazole is provided in an amount effective to reduce protein aggregates in the central nervous system. In a further embodiment, the disclosure provides use of aripiprazole for treating a polyglutamine disease in a subject in need thereof. Use of aripirazole in the manufacture of a medicament for use in treatment of neurodegenerative disease, polyglutamine disease, and/or MJD/SCA3 also is provided, as is aripiprazole for use in the treatment of neurodegenerative disease, polyglutamine disease, and/or MJD/SCA3.

In any of the methods of the disclosure, aripiprazole is optionally administered in an amount effective to decrease protein aggregates and/or HMW species of a mutant protein, such as mutant ATXN3, by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more, compared to baseline, untreated, or vehicle-treated subject.

In another aspect, the disclosure provides a method of reducing intracellular ATXN3 levels comprising contacting a cell with an effective amount of aripiprazole. In one aspect, the cell is a neuron or glial cell. In another aspect, the intracellular ATXN3 is mutant ATXN3 comprising an expanded polyglutamine tract compared to wild-type ATXN3. Optionally, a method of the disclosure comprises contacting a cell with an amount of aripiprazole effective to decrease the intracellular level of a mutant protein, such as mutant ATXN3, by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more, compared to an untreated or vehicle-treated cell.

In one aspect of the present methods, a therapeutically effective amount of aripiprazole, typically formulated in accordance with pharmaceutical practice, is administered to a subject in need thereof. In one aspect of the present methods, the subject is a human patient. A particular administration regimen for a particular subject will depend, in part, upon the condition of the subject, the amount of aripiprazole administered, the route of administration, and the cause and extent of any side effects. The amount administered to a subject in accordance with the invention should be sufficient to effect the desired response over a reasonable time frame. Dosage typically depends upon the route, timing, and frequency of administration. One of ordinary skill will appreciate that treating a neurodegenerative disease does not require complete eradication of the condition. Any beneficial physiologic response is contemplated, such as reduction, prevention, halting or delay of neuronal damage; decrease in levels of toxic proteins; increase in markers of protein degradation; alleviation or prevention/delay of neurological symptoms; increased longevity; and the like.

Purely by way of illustration, the methods of the present disclosure comprise administering, e.g., from about 0.1 mg/kg to about 15 mg/kg or more of aripiprazole based on the body weight of the subject, depending on the factors mentioned above. In some aspects, the dosage ranges from about 0.1 mg/kg to about 0.5 mg/kg, about 1 mg/kg to about 3 mg/kg, about 0.5 mg/kg to about 5 mg/kg, about 0.2 mg/kg to about 0.8 mg/kg, about 5 mg/kg to about 15 mg/kg, about 4 mg/kg to about 12 mg/kg, or about 0.1 mg/kg to about 2 mg/kg. For example, aripiprazole may be administered to a human patient in an amount from between about 1 mg to about 50 mg, for example, about 1 mg, about 5 mg, about 10 mg, about 15 mg, about 20 mg, about 25 mg, about 30 mg, about 35 mg, about 40 mg, about 45 mg, or about 50 mg. The dosage is administered as needed, for example, one to three times daily, every other day, twice a week, weekly, every two weeks, monthly, or less frequently. The treatment period will depend on the particular condition and may last one day to several days, weeks, months, or years. The above dosages are exemplary of the average case, but there can be individual instances in which higher or lower dosages are merited, and such are within the scope of this disclosure.

Suitable methods of administering aripiprazole, and pharmaceutically acceptable compositions thereof, are known in the art and are described in U.S. Pat. Nos. 7,053,092; 8,017,615, 8,759,350; and 9,125,939, incorporated herein by reference. In one aspect, the compound or composition is administered orally. In another aspect, the compound or composition is injected intravenously and/or intraperitoneally. For example, in certain circumstances, it will be desirable to deliver the composition through injection or infusion by intravenous, intraperitoneal, intracerebral (intra-parenchymal), intracerebroventricular, intraocular, intraarterial, intraportal, intralesional, intramedullary, intrathecal, intraventricular, or intranasal means; by controlled, delayed, sustained or otherwise modified release systems; or by implantation devices. Alternatively, aripiprazole is administered via implantation of a matrix, membrane, sponge, or another appropriate material onto which the compound has been absorbed or encapsulated. Where an implantation device is used, the device is, in one aspect, implanted into or on the surface of any suitable tissue or organ, and delivery of aripiprazole is, for example, via diffusion, timed-release bolus, or continuous administration. In one aspect, aripiprazole may be attached to a targeting moiety specific for delivery to a site in the central nervous system, such as an antigen binding protein including, but not limited to, antibodies, antibody fragments, antibody derivatives, antibody analogs, and fusion proteins, that bind, for example, a specific antigen on a neuron or glial cell.

The present disclosure will be more readily understood by reference to the following example, which is provided by way of illustration and is not intended to be limiting.

### Example

### Materials and Methods

Plasmids and Cell Culture.

Full-length ATXN3 (Accession number ABS29269) carrying either a normal or an expanded polyQ tract (Q26 or Q81) was subcloned in the vector pcDNA3.1 FLAG.firefly Luciferase. The mammalian expression plasmids pcDNA.FLAG.ATXN3Q26.Luc and pcDNA.FLAG.ATXN3Q81.Luc were confirmed by sequencing and shown to express N-terminal FLAG-tagged ATXN3Q26 or ATXN3Q81 fused with firefly Luciferase (Luc), respectively. These neomycin-resistant plasmids were transfected into HEK293 cells using Lipofectamine LTX (Invitrogen) during four hours, after which medium was replaced by growth medium (DMEM/10% fetal bovine serum (FBS)/1% penicillin/streptomycin (PS)). On the following day, medium was replaced for selection medium consisting of growth medium with 1000 mg/mL geneticin, which was shown to kill all the parent HEK293 cells in a killing curve carried out just before the transfection experiments. After one month of passage in selection medium, stably transfected clones for each cell line (293.ATXN3Q26Luc and 293.ATXN3Q81Luc) were pooled and frozen in liquid nitrogen. Stably transfected cell lines were maintained in selection medium except during treatment with specific compounds that were diluted in growth medium.

Small Molecule Screens.

For the primary screen, 2880 small molecules, including the Microsource Spectrum Collection of 2000 compounds including 1000 drugs (800 of which are FDA-approved), the NIH Clinical Collection (NCC) of 450 FDA-approved drugs, and another collection of 430 natural products with known biological activity, were tested. Stably transfected 293.ATXN3Q81Luc cells were plated in columns 1 to 22 of 384-well plates (2.5×10³ cells/well) in a final volume of 40 μL of DMEM/10% FBS/1% PS. Parental HEK293 cells were plated in columns 23 and 24, and a total of nine plates were incubated at 37° C./5% CO₂. Following 24 hours, columns 3 to 22 were spotted with 0.2 μL of library compounds in dimethyl sulfoxide (DMSO) (final concentration of 8 μM) using a Biomek FX laboratory automation workstation with high-density replication (pin tool). Columns 1-2 and 23-24 were spotted with 0.2 μL of DMSO, corresponding respectively to negative and positive controls. After 48 hours of incubation at 37° C./5% CO₂, 10 μL of STEADY-GLO Luciferase Assay System (Promega) was added to each well, and after a 10-minute incubation at room temperature, the activity of firefly Luciferase was measured in a PHERAstar plate reader (BMG Labtech). Actives were defined using the standard deviation (SD) values computed by the MScreen Database (Jacob et al. J Biomol Screen 17, 1080-1087 (2012)) for the negative controls (NC) on a plate by plate basis. Samples with a standard deviation by plate ≥3 were defined as actives. This criterion produced 162 active samples. Additional triage criteria excluded 28 molecules that were active in three or more additional Luciferase-based assays, 11 molecules that represented general promiscuity ((% assays ≥3 SD for NC)>30.0%), and 3 compounds showing black structure alert. A total of 120 small molecules were selected for further dose response titration. For the dose response screen (DRS), the 120 small molecules selected for dose response were screened in duplicate using eight serial 1:1 dilutions starting at 60 μM. Two sets of six plates were prepared in parallel following the same protocol described above. Forty-eight hours after compound addition, one set of plates was assayed for firefly Luciferase activity as described above. The other set of plates was assayed for cellular viability by adding 5 μL of AlamarBlue (Invitrogen) to each well, incubating 90 min at 37° C./5% CO₂, and measuring fluorescence (excitation 560 nm, emission 600 nm, cutoff 590 nm) in a Spectra Max M5 microplate reader (Molecular Devices). Nine of the ten selected compounds from the DRS were available for purchase from vendors and were acquired as fresh powders: artemether, CAS 71963-77-4 (A29361, Sigma Aldrich); aripiprazole, CAS 129722-12-9 (565444, AK Scientific); AM251, CAS 183232-66-8 (1117, Tocris Bioscience); cefamandole sodium, CAS 30034-03-8 (C7145, Sigma Aldrich); clotrimazole, CAS 23593-75-1 (C6019, Sigma Aldrich); mifepristone, CAS 84371-65-3 (M8046, Sigma Aldrich); monensin sodium, CAS 22373-78-0 (M5273, Sigma Aldrich); salinomycin sodium, CAS 55721-31-8 (anhydrous) (46729, Sigma Aldrich); tranilast, CAS 53902-12-8 (T0318, Sigma Aldrich).

Mouse Procedures and Treatment of YACMJD84.2 Mice with Aripiprazole.

YACMJD84.2 transgenic mice were housed in cages with a maximum number of five animals and maintained in a standard 12-hour light/dark cycle with food and water ad libitum. Genotyping was performed using DNA isolated from tail biopsy at the time of weaning, and genotypes of all studied mice were confirmed using DNA extracted from tails collected post-mortem. For mouse treatments, aripiprazole was dissolved in DMSO/Tween-80 in a 1:1 ratio and its pharmacokinetic parameters were determined for intraperitoneal (IP) injections: 12-week old wild type littermates from the YACMJD84.2 colony were IP injected with aripiprazole (15 mg/kg at 20 mL/kg in 96% saline/2% DMSO/2% Tween-80 as vehicle), and plasma and brain were collected at four time points after injection (0.5, 4, 8 and 24 hours; 2 females per group). For the subchronic treatment, a group of 12- to 14-week old hemizygous YACMJD84.2 (Q84) mice were injected (IP) daily with aripiprazole 15 mg/kg, 20 mL/kg or vehicle for 10 days (N mice per group=9 (5 females and 4 males)). Five hours after the final injection, mice were anesthetized with ketamine/xylazine and perfused transcardially with phosphate buffered saline (PBS) (for RNA and protein studies), and brains were immediately placed on dry ice and stored at −80° C. Another group of 12- to 14-week old Q84 mice were treated with aripiprazole or vehicle and sacrificed in the same way (N aripiprazole=10 (4 females and 6 males); N vehicle=7 (2 females and 5 males)), but the right brain hemisphere was stored at −80° C. and the left hemisphere was fixed in 4% paraformaldehyde (PFA) during at least 3 days, and then impregnated in 30% sucrose (for immunofluorescence).

Organotypic Mouse Brain Slice Cultures.

Sagittal brain slices (300 μm thick) from Q84 mice (8 to 9 weeks old) were prepared as previously reported (Shakkottai, V. G. et al. J Neurosci 31, 13002-13014 (2011)). Nine slices per brain were used in each set of cultures. After a quick wash in the corresponding medium, each slice was placed on a cell culture insert (0.4 μm pore size, 30 mm diameter (Millipore)), which was previously placed on a well (6-well plate) containing 1.2 mL of culture medium (50% MEM with Earle's salts, 25% horse serum, 25% Hanks' balanced salts solution, 25 mM Hepes, 2 mM L-glutamine, 6.5 mg/mL glucose) containing a specific compound or its vehicle. After 48 hours of incubation at 37° C./5% CO₂, brain slices were assessed for ATXN3 levels by immunoblotting or immunofluorescence and for cell viability. For immunoblotting, each slice was macrodissected into separate cerebellum and brainstem that were immediately frozen at −80° C. in 80 μL and 150 μL of cold RIPA buffer containing protease inhibitors (Complete, Roche Diagnostics), respectively. Cell viability was assessed on treated slices by incubation in propidium iodide (PI) 1 μg/mL in culture medium for 1 hour. Slices were then mounted in PROLONG Gold Antifade Reagent (Invitrogen) and imaged using a FV500 Olympus confocal microscope.

Western Blotting.

Protein lysates from cell cultures, mouse brain slices or mouse brains were obtained by lysis in RIPA buffer containing protease inhibitors (Complete, Roche Diagnostics), followed by sonication and centrifugation. The supernatants (soluble protein fractions) were collected, total protein concentration was determined using the BCA method (Pierce) and then stored at −80° C. Total proteins (50 μg from cell and slice cultures or 75 μg from brain regions of mice) were resolved in 10% SDS-PAGE gels, and corresponding PVDF membranes were incubated overnight at 4° C. with primary antibodies: mouse anti-ATXN3 (1H9) (1:2000; MAB5360, Millipore), goat anti-Luciferase (1:500; G7451, Promega), mouse anti-FLAG (M5) (1:500; IB13091, Sigma), rabbit anti-LC3 (1:500; PM036, MBL International Corporation), mouse anti-HSP90β (1:1000; ADI-SPA842, Enzo Life Sciences), rabbit anti-HSP90α (1:1000; ab2928, Abcam), mouse anti-HSP70 (1:500; SPA810, Enzo Life Sciences), rabbit anti-HSP40 (1:1000; #4868, Cell Signaling), rabbit anti-HSP25 (1:1000; ADI-SPA801, Enzo Life Sciences), rabbit anti-HSF1 (1:1000, ADI-SPA-901, Enzo Life Sciences), rabbit anti-ubiquitin (1:1000, Z 0458, Dako), rabbit anti-RAD23A (1:5000; TA307264, Origene), rabbit anti-RAD23B (1:2000; A302-306A, Bethyl Labs), rabbit anti-α-Tubulin (11H10) (1:10000; #2125, Cell Signaling), and mouse anti-GAPDH (1:1000; MAB374, Millipore). Pellets corresponding to insoluble protein fractions were resuspended in 100 μL of Laemmli buffer 2× and boiled for 10 min. Insoluble proteins were then quantified, and 100 μg were loaded in a filter trap assay apparatus and transferred to a nitrocellulose membrane (0.45 μm pore) that was incubated overnight with rabbit anti-MJD antibody (1:5000) at 4° C. Bound primary antibodies were visualized by incubation with a peroxidase-conjugated anti-mouse or anti-rabbit secondary antibody (1:10000; Jackson Immuno Research Laboratories) followed by treatment with the ECL-plus reagent (Western Lighting, PerkinElmer) and exposure to autoradiography films. Band intensity was quantified by densitometry using ImageJ.

Quantitative Reverse Transcriptase (RT)-PCR.

Total RNA from brainstem fractions of mice treated with aripiprazole or vehicle was obtained by an initial extraction using Trizol Reagent (Invitrogen) followed by purification using the RNEASY mini kit (Qiagen) following the manufacturer's instructions. Reverse transcription of 1.5 μg of total RNA per sample was performed using the iScript cDNA synthesis kit (Bio-RAD). Human ATXN3 and mouse Atxn3, Drd2, 5HT1A, 5HT2A and Gapdh (housekeeping) transcript levels were accessed by quantitative real-time PCR. Relative gene expression was determined using the ΔΔC_{T} method, normalizing for Gapdh mRNA levels.

Immunofluorescence, Quantification of Nuclear ATXN3 and Counting of ATXN3-Positive Puncta.

Brains from mice perfused with 4% PFA were post-fixed overnight at 4° C. in the same fixative, immersed in 30% sucrose/PBS, and sectioned on a sledge microtome (SM200R Leica Biosystems). Free-floating 40 μm sagittal sections were collected and stored in cryoprotectant solution at −20° C. Brain sections from treated mice or brain slices from organotypic cultures processed for immunofluorescence were initially subjected to antigen retrieval and incubated using the Vector MOM immunodetection kit (Vector Laboratories). For double or single immunofluorescence, sections were incubated with mouse anti-ATXN3 (1H9) (1:1,000; MAB5360 Millipore) and rabbit anti-NeuN (1:1,000; ABN78 Millipore), and then incubated with the corresponding secondary Alexa Fluor 488 and/or 568 antibodies (1:1,000; Invitrogen). All sections were then stained with 4,6-Diamidino-2-phenylindole dihydrochloride (DAPI, Sigma), mounted with PROLONG Gold Antifade Reagent (Invitrogen), and imaged using a FV500 Olympus confocal microscope. Single-plane images of ventral pons from mice treated with aripiprazole or vehicle were acquired using a 60×W objective. Neuronal nuclear ATXN3 was quantified using ImageJ by quantifying ATXN3 fluorescence in DAPI and NeuN double-positive nuclei. Corrected total cell fluorescence (CTCF) was obtained by measuring ATXN3 fluorescence in 15 nuclei/image field/mouse and normalizing for nuclei area using the equation CTCF=fluorescence intensity−(nucleus area×mean fluorescence of background readings). ATXN3 positive puncta were quantified using ImageJ by assessing particle fluorescence (applying the triangle threshold) in the same image fields described above.

Treatment of MJD Flies with Aripiprazole.

Drosophila stocks were reared on standard cornmeal media at 25° C. in diurnal environments with ˜60% humidity. New fly lines were generated using the site-specific pHiC31 integration system (Keravala, A. & Calos, M. P. Methods in molecular biology 435, 165-173 (2008)) into site attP2 of the third chromosome of the fruit fly. Full-length human ATXN3 cDNA with 77 CAG was cloned into pWalium10-moe (Perrimon Lab, Harvard Medical School). Flies that carry ATXN3 in site attP2 or the empty vector control into the same site as the most isogenic control line were generated. Injections were done into y[1], w[*]; +; attP2 (landing site). For aripiprazole treatments, instant media (Nutri-Fly Instant, Genesee Scientific) was prepared with vehicle (1:1 DMSO-Tween-80, Sigma Aldrich) or vehicle with compound (aripiprazole, AK Scientific) at 50 μM. Adult flies of the following genotypes, w[*]; sqh-Gal4/+; UAS-ataxin-3Q77/+ or w[*], sqh-Gal4/+; Empty-Vector-Control/+ were switched from standard media to instant media as soon as they eclosed from their pupal case (day 0), and thereafter maintained on instant media with vehicle compound. Media was switched every three days, and death was noted daily.

Recombinant Protein Expression and Purification.

pGEX-6P1 plasmids encoding GST-ATXN3Q26 and GST-ATXN3Q55 were transformed in Rosetta (DE3) BL21 cells. Colonies grown overnight in LB/ampicillin/chloramphenicol plates at 37° C. were resuspended in 150 mL of the same selection medium and incubated at 37° C., 230 rpm for 2 hours. Bacterial cultures of 1 L of medium were prepared by inoculating 50 mL of the pre-culture and incubating at 37° C., 230 rpm until reaching an OD (600 nm) of 0.6 to 0.8. Expression of fusion proteins was then induced by adding 1 mM isopropyl-1-thio-D-galactopyranoside (IPTG) for 3 hours at 37° C. Cells were collected by centrifugation and stored at −20° C. Cell pellets were resuspended in 20 mL of lysis buffer (150 mM NaCl, 50 mM Tris (pH 7.5), 0.5% NP-40, protease inhibitors (COMPLETE, Roche Diagnostics), PMSF, lysozyme), incubated on ice for 30 min, additionally lysed by sonication, and finally centrifuged at 15000 rpm, 20 min, at 4° C. The supernatants were collected and incubated with 1 mL of glutathione Sepharose beads (GE Healthcare) for 3 hours at 4° C., with rotation. Beads were then washed in cold PBS, resuspended in 5 mL of PBS containing 40 μL of Prescission Protease (2000 units/mL, GE Healthcare) and incubated at room temperature (RT) for 15 min. Cleaved ATXN3 was collected in the supernatant after centrifugation at 700×g for 5 min. Additional ATXN3 was recovered from beads after 3 subsequent steps of resuspension in PBS, incubation at RT and centrifugation. ATXN3Q26 and ATXN3Q55 solutions were concentrated in Ultra-15 centrifugal filter units (Amicon) and proteins were purified by fast protein liquid chromatography (FPLC) using a Superdex-200 column (GE healthcare) and 50 mM Na₂PO₄, 100 mM NaCl, 1 mM NaN₃ (pH 7.4) buffer. Chromatography fractions were analyzed by SDS-PAGE, and the ones containing pure proteins were concentrated and protein concentration was determined in the Nanodrop (Thermo Scientific) by absorption at 280 nm.

ATXN3 Fibril Formation.

For the Thioflavin T assay, solutions of ATXN3Q26 or ATXN3Q55 at a final concentration of 10 μM were prepared in the presence of aripiprazole (400 μM) or vehicle in 25 mM Na₂PO₄, 200 mM NaCl, 10 μM Thioflavin T, pH 7.4. Samples and blank control (only buffer) (110 μL) were dispensed in each well of a Black/Clear flat bottom 96-well plate (Corning), which was sealed and incubated at 37° C. with agitation in a FLUOstar Omega (BMG Labtech Inc) plate reader. Fluorescence of three replicates of each sample was measured every 10 min for up to 5 days. The emission and excitation wavelengths of the filter were 440 nm and 490 nm, respectively, and readings were taken using 90% gain adjustment. Values for protein solutions were normalized to readings of blank buffer.

Native Gel Electrophoresis.

ATXN3Q26 and ATXN3Q55 protein solutions (10 μL of each sample) were monitored before and after the fibrillation assay in the presence of aripiprazole or vehicle using a NativePAGE Novex Bis-Tris gel system (Life Technologies) following the manufacturer's protocol.

Transmission Electron Microscopy.

Negatively stained specimens for transmission electron microscopy (TEM) were prepared by applying 5 μL of sample on hydrophilic 400 mesh carbon-coated support films mounted on copper grids (Ted Pella, Inc.). The samples were allowed to adhere for 4 min, rinsed twice with distilled water, and stained for 60 to 90 sec with 1% uranyl acetate (Ted Pella, Inc.). Samples were imaged at an accelerating voltage of 60 kV in a Philips CM-100 microscope.

Statistical Analysis.

Levels of proteins and transcripts, and fluorescence intensity were compared using Student's t-test (comparison of two groups) whenever distributions were normal and homogeneous. In the other cases, comparisons were assessed using the non-parametric Mann-Whitney U test. Fly survival was analyzed using Kaplan-Meier curves and the Log-rank Mantel-Cox test was used to compare survival curves. A p<0.05 was considered statistically significant in all analyses. Data were analyzed using IBM SPSS Statistics 22 software.

### Results

Identification of Small Molecules that Reduced Levels of Mutant ATXN3 in a Cell-Based Assay.

Cell-based assays to identify small molecules that reduce levels of ATXN3 were developed. HEK293 cell lines that stably express FLAG-tagged full-length human ATXN3 with a normal or expanded polyQ tract (Q26 or Q81) fused to firefly Luciferase (Luc) termed ATXN3Q26.Luc and ATXN3Q81.Luc, respectively, were generated. In the cell assay, levels of ATXN3/Luciferase fusion proteins were measured by chemiluminescence. The ATXN3Q81.Luc cells were used in a 384-well format to screen 2880 small molecules, including 1250 FDA-approved drugs. The molecules, comprising 2402 unique chemical structures, were screened at [8 μM] for 48 hours of treatment (average plate Z factor 0.81). Among 162 actives with a standard deviation by plate of ≥3, 120 compounds were selected for dose-response screens (DRSs). Luminescence and viability DRSs were run in parallel using duplicates of 8 concentrations for each molecule (range 0.47 μM to 60 μM). Ten structurally diverse compounds met criteria for follow up screens (IC50<100 μM, viability >70%, and luminescence inhibition ≥20%), nine of which were available for purchase from vendors.

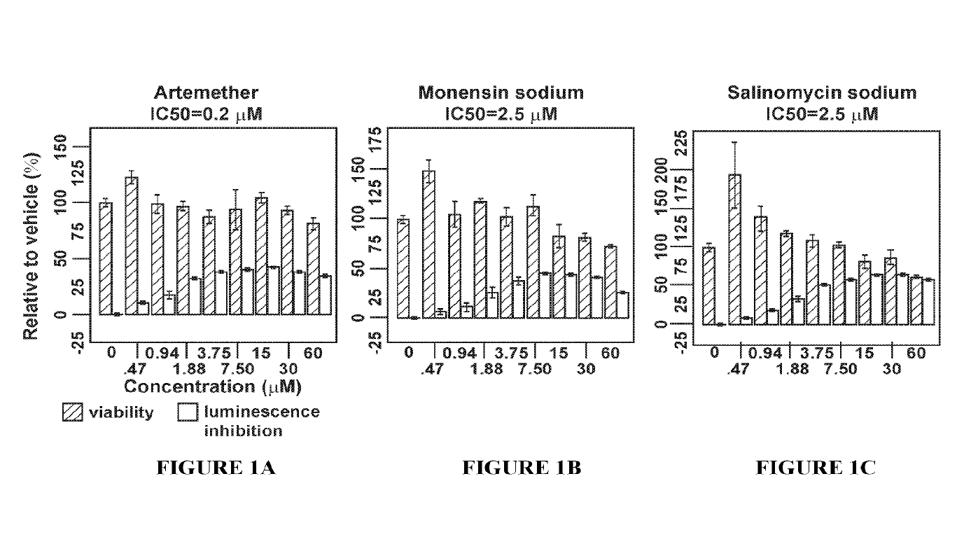

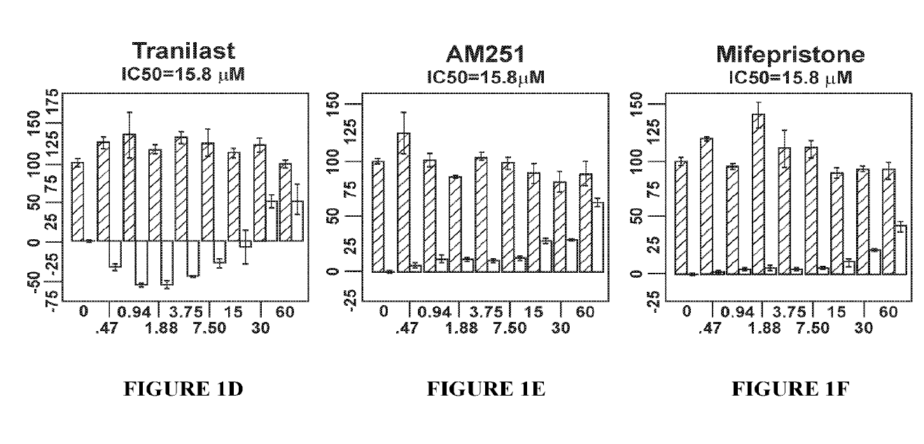

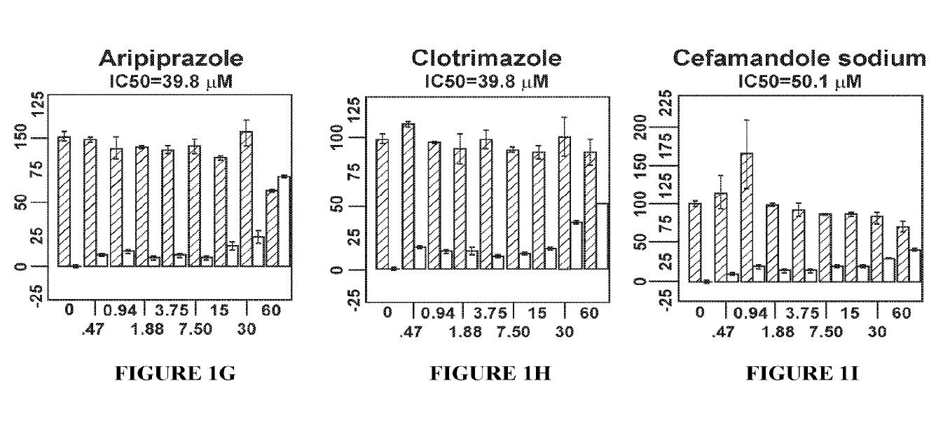

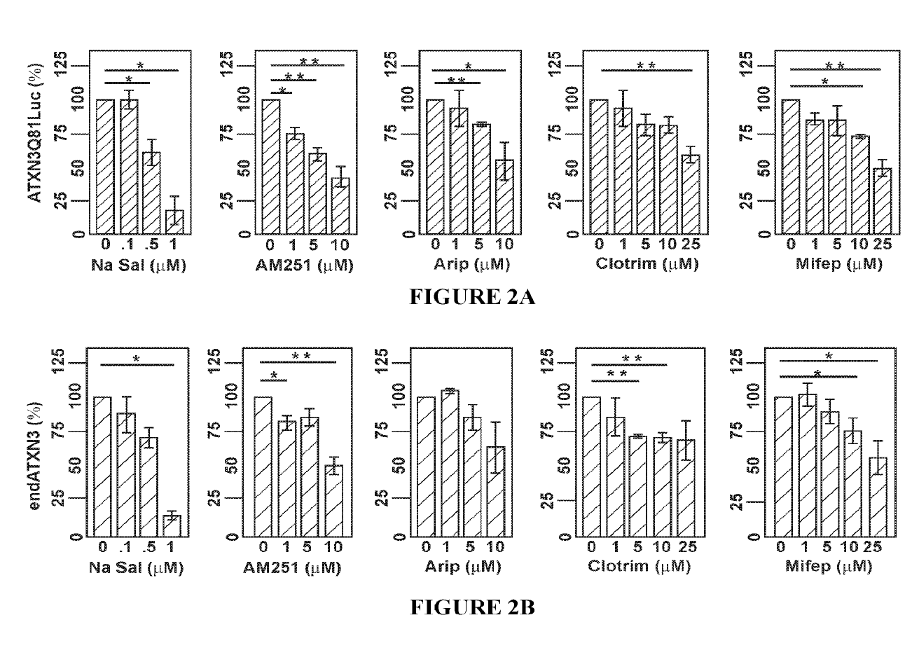

The nine compounds, showing an IC50 that ranged from 0.2 to 50.1 μM (FIG. 1A to FIG. 1I), were tested in independent dose-response experiments in both 293.ATXN3Q26Luc and 293.ATXN3Q81Luc cell lines, with the efficacy of each molecule assessed by measuring ATXN3 levels by immunoblotting. Five of the nine tested compounds (salinomycin sodium, AM251, aripiprazole, clotrimazole and mifepristone) were confirmed to decrease levels of ATXN3Q81.Luc fusion protein (FIG. 2A). The compounds reduced ATXN3 levels in a polyQ-length independent manner, as they also reduced the amount of non-expanded ATXN3Q26Luc (data not shown) and of endogenous ATXN3 (FIG. 2B), further indicating that they could act on ATXN3 expressed at physiological levels.

Aripiprazole, AM251 and Salinomycin Sodium Reduced Human Mutant ATXN3 in Organotypic Brain Slice Cultures from YACMJD84.2 Transgenic Mice.

To assess the efficacy of the five compounds to reduce ATXN3 levels in the mammalian brain, secondary screens in organotypic brain slice cultures derived from hemizygous YACMJD84.2 (Q84) transgenic mice were performed. These mice harbor the full-length human ATXN3 disease gene with an expanded repeat of 84 CAGs (Cemal et al. Hum Mol Genet 11, 1075-1094 (2002)) and therefore express all human pathogenic ATXN3 isoforms, the precise target in MJD/SCA3 patients.

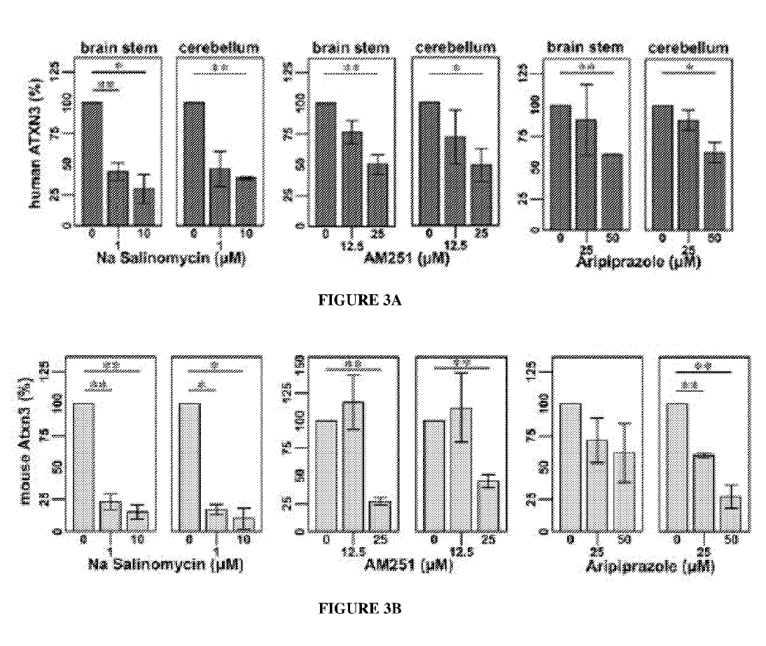

In sagittal brain slices treated for 48 hours, aripiprazole, AM251 or salinomycin sodium reduced levels of human mutant ATXN3Q84 and mouse Atxn3 in the cerebellum and brainstem, the two most affected brain regions in MJD patients (FIG. 3A and FIG. 3B). In contrast, clotrimazole and mifepristone did not alter ATXN3 levels. Treatments did not affect slice viability assessed by propidium iodide uptake except for salinomycin sodium at 10 μM. Aripiprazole (PubChem CID 60795) is an atypical antipsychotic agent; AM251 (PubChem CID 2125) is a cannabinoid receptor 1 (CB1) antagonist; and salinomycin sodium (PubChem CID 5748657) is an antibacterial and coccidiostat compound with selective toxicity against cancer stem cells. Aripiprazole was selected for further in vivo testing in fly and mouse models of MJD/SCA3.

Aripiprazole Delayed Onset of Mutant Ataxin-3-Mediated Toxicity in MJD/SCA3 Flies.

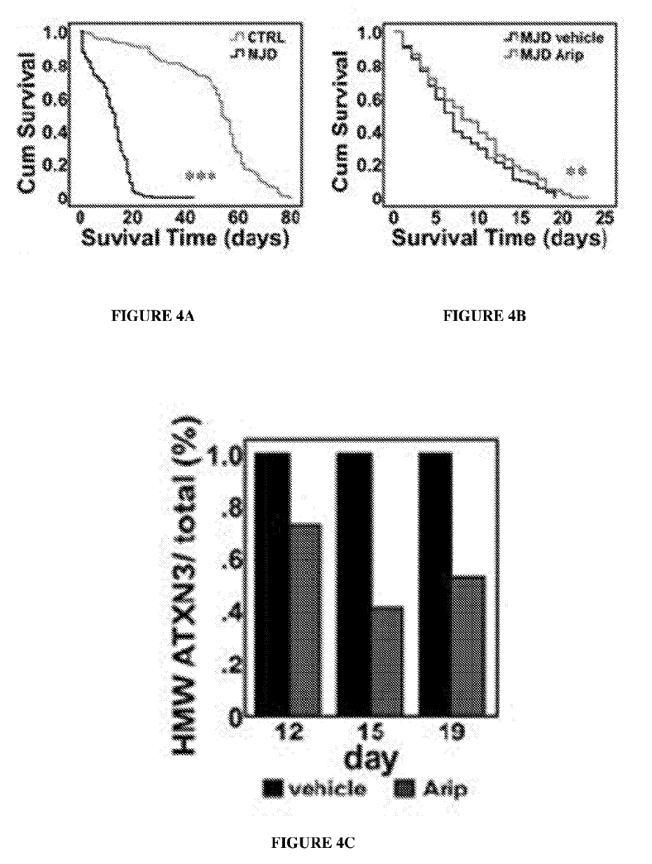

To test the efficacy of aripiprazole in vivo, novel transgenic Drosophila lines that express full-length human ATXN3 with a pathogenic polyQ tract of 77 glutamines (MJD) through the Gal4-UAS system of targeted expression were generated. When the sqh-Gal4 driver expresses mutant UAS-ATXN3 throughout Drosophila, MJD/SCA3 flies had a markedly shortened lifespan (mean survival 11.5 days ±0.376) compared to flies containing the empty vector control (CTRL) (mean survival 50.5 days ±1.041) (FIG. 4A) inserted at the same chromosomal site as ATXN3.

To mirror the treatment in MJD/SCA3 patients, which would start in adult life, treatment of MJD/SCA3 flies started upon eclosion from the pupal case (day 0 in FIG. 4A and FIG. 4B) by placing them in quick formula food containing either vehicle or aripiprazole (50 μM, the effective dosage in mouse brain slice cultures). At least 200 flies in groups of 9 to 17 flies per treatment vial were monitored. Aripiprazole-treated MJD/SCA3 flies showed increased mean survival of 1.3 days (9.0±0.367 days) compared with vehicle-treated MJD/SCA3 flies (7.7±0.343) (FIG. 4B). ATXN3 immunoblotting of protein lysates from fly heads (10 per group) revealed that aripiprazole decreased high molecular weight (HMW) ATXN3 species to 0.73, 0.41 and 0.53 of levels in vehicle-treated flies at days 12, 15 and 19, respectively, concomitant with the increased longevity (FIG. 4C).

MJD/SCA3 Transgenic Mice Treated with Aripiprazole Showed Decreased Levels of Soluble Mutant ATXN3 in Brain.

The ability of aripiprazole to decrease levels of pathogenic ATXN3 in vivo in brains of Q84 mice was assessed. Twelve-week-old Q84 mice (9 mice per group, comprising 5 females and 4 males) were treated for 10 days with daily intraperitoneal injections of vehicle or aripiprazole (15 mg/kg, the maximum tolerable dose reported in chronically treated mice) (Madhavan et al. J Neurosci 33, 12329-12336 (2007)). The aripiprazole was rapidly absorbed, showing maximal concentration in the brain 30 min post-injection. Five hours after the final injection on day 10, mice were sacrificed and brains collected for total protein and RNA extraction from different regions: brainstem, cerebellum, cervical spinal cord, and forebrain.

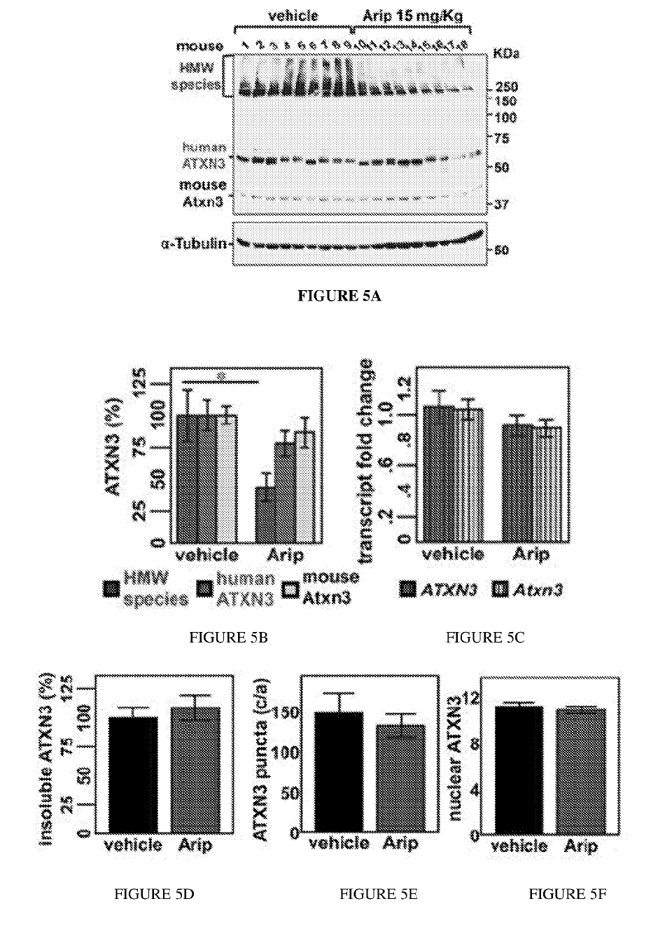

Immunoblot analysis of soluble lysates revealed a reduction in HMW ATXN3 species in the brainstem from aripiprazole-treated mice, whether male or female, to 44% of levels in vehicle-treated mice (FIG. 5A and FIG. 5B). A similar reduction of HMW ATXN3 was observed in the cerebellum, spinal cord, and forebrain. A trend toward decreased levels of monomeric human ATXN3 and endogenous mouse Atxn3 was also observed (FIG. 5A and FIG. 5B), indicating that longer treatments with aripiprazole could decrease all forms of ATXN3. A potential effect of aripiprazole at the ATXN3 transcription level was ruled out, as human ATXN3 and mouse Atxn3 transcript levels were similar in brainstems from aripiprazole and vehicle-treated mice (FIG. 5C).

As aripiprazole preferentially decreased HMW ATXN3 levels from soluble protein fractions, the effect on levels of insoluble ATXN3 was assessed. Filter trap analysis of insoluble fractions, from the same brainstem samples described above, revealed no differences of insoluble ATXN3 between the two treatments (FIG. 5D). Furthermore, anti-ATXN3 immunofluorescence of ventral pons of Q84 mice treated with aripiprazole showed similar abundance of ATXN3-positive puncta as in vehicle-treated mice (FIG. 5E). Because mutant ATXN3 accumulates and tends to form insoluble aggregates in the nucleus (Chai et al. Proc Natl Acad Sci USA 99, 9310-9315 (2002)), ATXN3 levels in neuronal nuclei of ventral pons were assessed by immunofluorescence. Fluorescence quantification revealed no differences in ATXN3 nuclear levels between the two treatments (FIG. 5F). Comparing with control mice, pons from aripiprazole-treated mice showed a non-significant trend towards a decrease of total ATXN3 fluorescence, which corresponded to the apparent slight reduction of cytoplasmic ATXN3 fluorescence in these mice. ATXN3 soluble aggregates observed by immunoblotting were not detectable by regular immunofluorescence. In summary, aripiprazole was effective to decrease soluble ATXN3, in particular the HMW species observed by immunoblotting, but not more insoluble ATXN3 species detected by the filter-trap assay or immunofluorescence (puncta).

The mechanism of action of aripiprazole is complex and not fully understood. Aripiprazole exerts its efficacy as an atypical antipsychotic by partial agonism at dopamine D2 receptors (D2Rs) and serotonin 5-HT1A receptors together with antagonism at serotonin 5-HT2A receptors. As aripiprazole has been shown to increase dopamine receptor D2 (Drd2) mRNA levels in the ventral tegmental area (VTA) of rats (Han et al. The international journal of neuropsychopharmacology/official scientific journal of the Collegium Internationale Neuropsychopharmacologicum 12, 941-952 (2009)), transcript levels of Drd2 and the other main target receptors, 5HT1A and 5HT2A, in aripiprazole-treated mice were assessed. Indeed, aripiprazole was able to engage its targets by increasing Drd2 and 5HT2A transcripts in the brainstem of Q84 mice. This effect was mediated by aripiprazole because Drd2, 5HT1A and 5HT2A transcripts were similarly abundant in brainstems of 12-week-old Q84 mice and littermate wild type controls.

Aripiprazole Did not Interfere with Fibrillation of ATXN3 In Vitro.

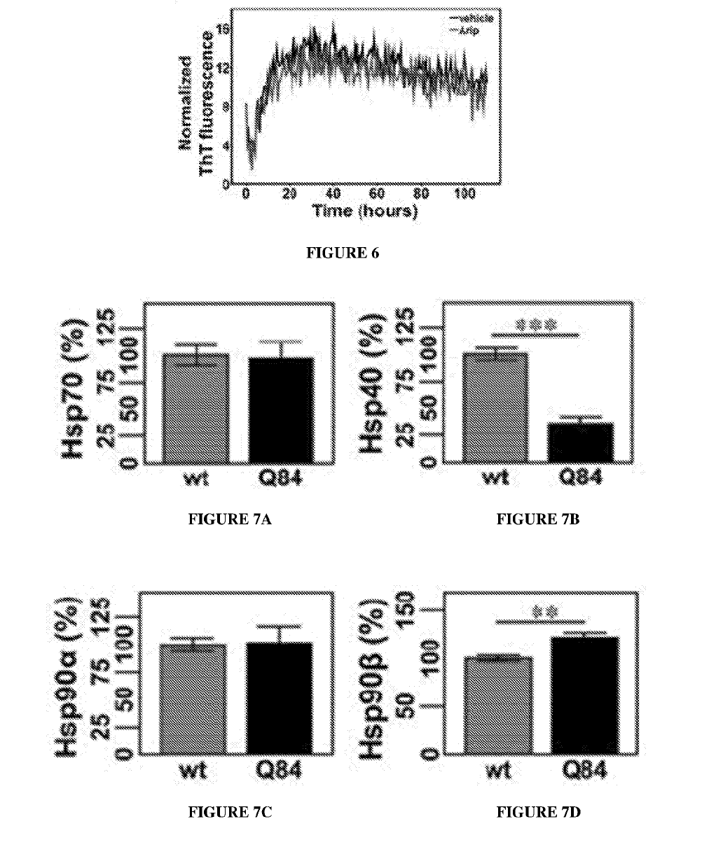

Because aripiprazole was effective in reducing soluble aggregates of ATXN3 in vivo, the ability of aripiprazole to directly modulate ATXN3 fibril formation was investigated. To test this possibility, fresh recombinant ATXN3 carrying a modestly expanded glutamine tract (ATXN3Q55) was incubated with aripiprazole or vehicle in the presence of Thioflavin T (ThT) and the change of fluorescence that occurs upon incorporation into amyloid-like fibrils was monitored. The kinetics of ATXN3Q55 fibril formation was identical in the presence or absence of aripiprazole (FIG. 6). Similarly, aripiprazole did not interfere with fibril formation by normal (i.e., nonpathogenic) ATXN3Q26. Corroborating this result, native PAGE analysis of samples at the end of the ThT assay revealed no differences in HMW or other species of ATXN3Q55 in the presence of aripiprazole. Furthermore, imaging using electron microscopy revealed ATXN3 spheroidal particles and short chains in both the presence or absence of aripiprazole.

Aripiprazole Altered Components of Protein Homeostasis Machinery in MJD/SCA3 Transgenic Mouse Brains.

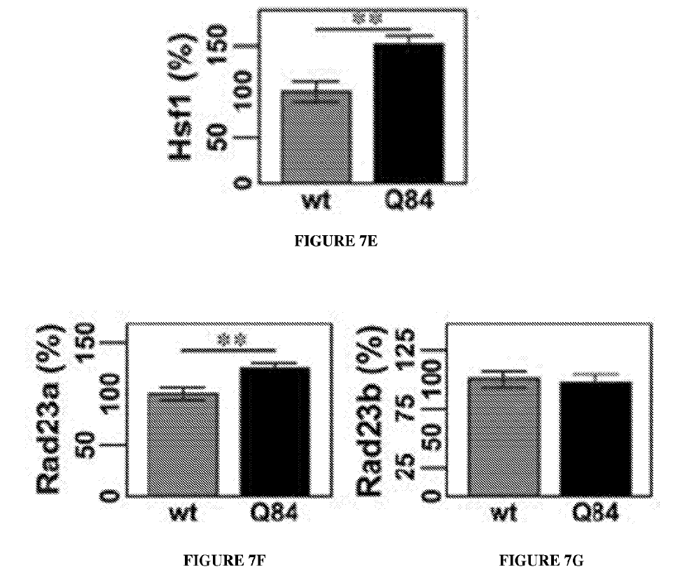

While aripiprazole did not directly modulate ATXN3 fibril formation in vitro, the hypothesis that it decreases soluble aggregates of ATXN3 by regulating key components of cellular protein homeostasis in vivo was investigated. Levels of such components in Q84 mice were first assessed. Brainstem lysates from 12-week old Q84 mice showed dysregulated levels of several components of chaperone machinery compared to wild type littermate controls (wt). Hsp40 was decreased (36% of control) (FIG. 7A), whereas Hsp90β (FIG. 7D) and Hsf1 (FIG. 7E) were increased (122% and 152% of control, respectively). Reduction of Hsp40 levels (FIG. 7B) appeared to be a consistent marker of mutant ATXN3 pathogenesis in MJD/SCA3 mouse brains as previously reported (Chou et al. Neurobiol Dis 31, 89-101 (2008)). On the other hand, increased levels of Hsp90β and Hsf1 in Q84 brainstems are also consistent with the fact that Hsp90 inhibition and activation of Hsf1 pathway led to improved phenotypes of MJD/SCA3 animal models (Silva-Fernandes, A. et al. Neurotherapeutics 11, 433-449 (2014); Teixeira-Castro et al. Hum Mol Genet 20, 2996-3009, (2011)).

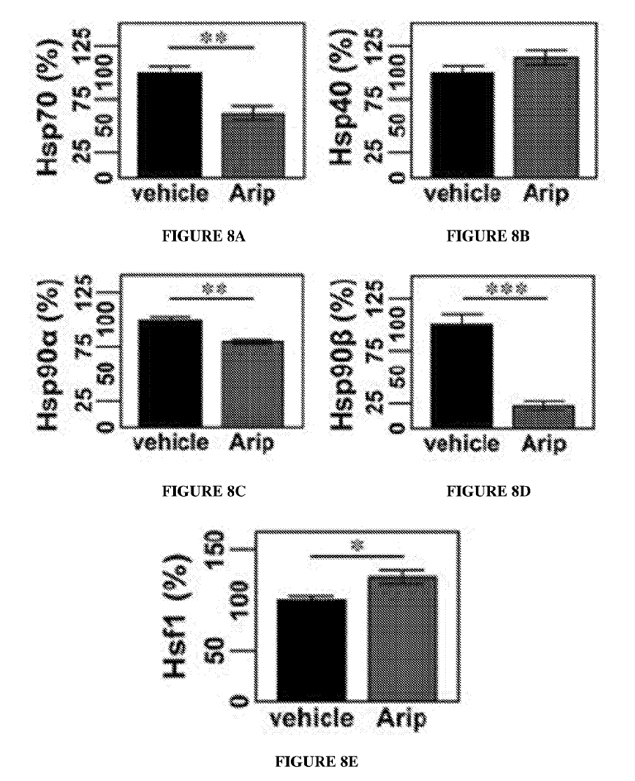

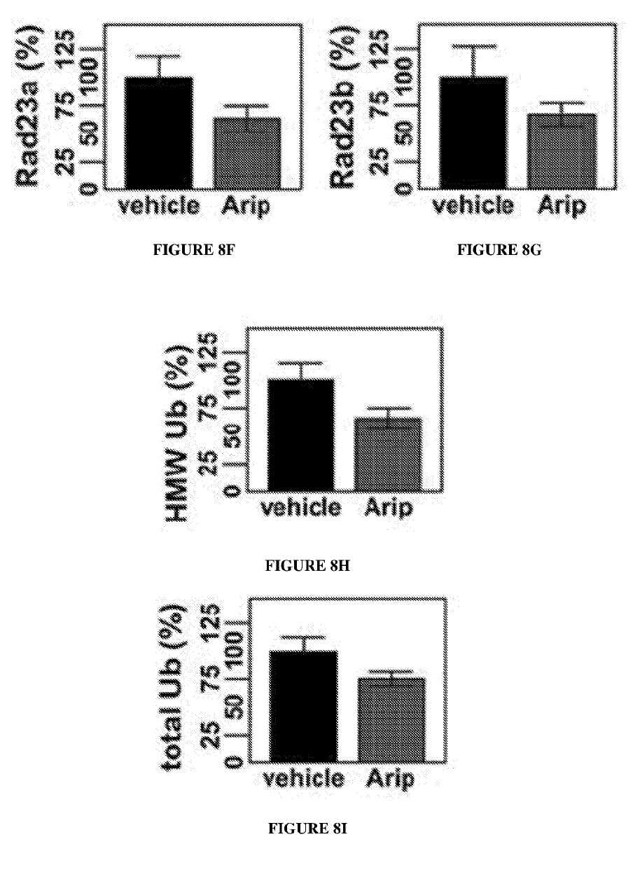

Analysis of soluble fractions from the brainstems of aripiprazole-treated Q84 mice revealed decreased levels of Hsp70 to 62% (FIG. 8A), Hsp90a to 79% (FIG. 8C) and Hsp90β to 22% (FIG. 8D), compared with vehicle-treated mice. No differences were observed in levels of Hsp40 (FIG. 8B). Heat shock transcription factor (Hsf1) was increased in samples from aripiprazole-treated mice (FIG. 8E), which could be related to the observed marked reduction of cytosolic forms of Hsp90, in particular Hsp90β.

Macroautophagy and the proteasome have been implicated in degrading mutant ATXN3 (Menzies et al. Brain 133, 93-104 (2010); Nascimento-Ferreira et al. Brain 134, 1400-1415 (2011); Jana. et al. J Biol Chem 280, 11635-11640 (2005); Matsumoto et al. EMBO J 23, 659-669 (2004)). While treatment differences in LC3I, LC3II or LC3II/LC3I ratio were not observed, a trend toward decreased levels of Rad23a and Rad23b, which are ATXN3 interactors that prevent its proteasomal degradation (Blount et al. Nat Commun 5, 4638 (2014)), was noted in aripiprazole-treated mice (FIG. 8F and FIG. 8G). As Rad23a levels were increased in Q84 mice (FIG. 8F), the aripiprazole-mediated response restored Rad23a abundance to normal (lower) levels and consequently promote ATXN3 proteasomal degradation. In summary, subchronic treatment of Q84 mice with aripiprazole appeared to affect proteostasis by altering levels of molecular chaperones and ATXN3 interactors implicated in its stability/degradation.

### DISCUSSION

An unbiased approach to screen small molecules was used to identify agents that promote reduction of mutant ATXN3 levels. The cell-based assays displayed excellent signal to noise properties (average plate Z-factor=0.81) and high reproducibility in the screen of 2880 small molecules. Efficacy of selected small molecules identified by the assay was first confirmed ex vivo using brain slice cultures from MJD/SCA3 transgenic mice (Q84) expressing the full-length human disease ATXN3 gene, and subsequently in vivo in MJD/SCA3 flies and in the same MJD/SCA3 mice. The ATXN3Q81.Luc cell line proved to be a robust assay for this initial, as well as for future, high-throughput screens to identify modulators of ATXN3 abundance.

Five of nine compounds selected from dose response screens were confirmed to be effective in independent testing using fresh compound powders and immunobloting for ATXN3 as the readout. Among these five compounds, salinomycin sodium, AM251, and aripiprazole reduced levels of human mutant ATXN3 in brainstem and cerebellar areas of brain slices from Q84 mice, which are the precise target regions in MJD/SCA3 patients. All these molecules interfered with intracellular Ca²⁺ levels either by binding to membrane receptors (AM25152 and aripiprazole), or by acting as an ionophore in cell membranes (salinomycin54). Calcium homeostasis is indeed dysregulated in MJD/SCA3 models. Strategies to re-establish Ca²⁺ balance by treating MJD/SCA3 transgenic mice with dantrolene (stabilizer of intracellular Ca²⁺ signaling), or by down-regulating PIK1 (an ATXN3 interactor that regulates the transport of ion channel subunits involved in calcium homeostasis) in MJD/SCA3 flies ameliorated several disease signs in these models (Chen et al. J Neurosci 28, 12713-12724 (2008); McGurk, L. & Bonini, N. M. Hum Mol Genet 21, 76-84 (2012)). At the cellular level, it is known that regulation of Ca²⁺ levels to decrease ATXN3 proteolysis correlates directly with a reduction in ATXN3 aggregation (Haacke et al. J Biol Chem 282, 18851-18856 (2007); Koch et al. Nature 480, 543-546 (2011); Simoes et al. Brain 135, 2428-2439 (2012)). Salinomycin, AM251, and aripiprazole interference with intracellular Ca²⁺ balance could in turn modulate the stability/clearance of mutant ATXN3.

In further in vivo testing, aripiprazole decreased mutant ATXN3-mediated toxicity in MJD flies by increasing survival, which correlated with the observed reduction of HMW ATXN3 species in these flies. In Q84 mice, a 10-day course of treatment with aripiprazole led to a reduction of soluble ATXN3, in particular the HMW (mutant/aggregated) species. While the relative toxicity of soluble versus insoluble polyQ protein aggregates is still debated, soluble oligomeric intermediates reside more upstream in the pathogenic cascade and are thought to be a critical toxic species in various neurodegenerative diseases (Williams, A. J. & Paulson, H. L. Trends Neurosci 31, 521-528 (2008)).

The fact that aripiprazole did not have a direct effect on ATXN3 fibrillation suggested that its protective effect was elicited extracellularly via dopaminergic and serotonergic signaling. While aripiprazole could potentially modulate intracellular Ca²⁺ levels to lessen ATXN3 proteolysis and consequent aggregation, the drug affected the levels of select PQC proteins in a manner that favors degradation of mutant ATXN3 by the proteasome. Rad23a and Rad23b are known to interact with ATXN3 and prevent its degradation by the proteasome. Because increased levels of Rad23a were observed in the brainstem of Q84 mice, proteasomal degradation of ATXN3 could be reduced in Q84 mouse brains. Observations in mice treated with aripiprazole were consistent with the drug increasing proteasomal clearance of ATXN3: (1) decreased levels of Rad23a and Rad23b, which are expected to increase ATXN3 accessibility to the proteasome; and (2) decreased levels of Hsp70, which could increase the targeting of misfolded mutant ATXN3 to the proteasome.

Molecular chaperones are key PQC components that ensure proper protein folding and help target misfolded proteins for degradation. Indeed, MJD/SCA3 transgenic mice showed altered levels of important components of the molecular chaperone machinery in the brainstem, namely, reduced Hsp40 and increased Hsp90β and Hsf1. Treatment of MJD/SCA3 mice with aripiprazole decreased levels of Hsp90a and Hsp90β, which could explain the observed further increase in Hsf1 abundance.

In conclusion, using a combination of in vitro, ex vivo and in vivo assays in human cell, mouse and fly models, aripiprazole was identified as a therapeutic agent for MJD/SCA3. Because aripiprazole reduced levels of oligomeric forms of mutant ATXN3, the drug would be effective in reducing other aggregate-prone proteins and therefore useful for treating a host of neurodegenerative proteinopathies.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed:

1. A method of treating a neurodegenerative disease in a subject in need thereof comprising administering a therapeutically effective amount of aripiprazole in an amount effective to reduce protein aggregates in the central nervous system.

2. The method of claim 1, wherein the subject has a neurodegenerative disease selected from the group consisting of Alzheimer's disease, Parkinson's disease, amyotrophic lateral sclerosis (ALS), frontotemporal dementia, spinocerebellar ataxia (SCA) type 1, SCA type 2, SCA type 6, SCA type 7, SCA type 17, Machado-Joseph disease/SCA type 3 (MJD/SCA3), Huntington's disease, dentatorubral pallidoluysian atrophy (DRPLA), and spinal and bulbar muscular atrophy, X-linked 1 (SBMA).

3. The method of claim 1 or claim 2, comprising administering aripiprazole in an amount effective to decrease protein aggregates in an area of the central nervous system of the subject selected from the brainstem, cerebellum, spinal cord, forebrain, and combinations thereof.

4. A method of treating a polyglutamine disease in a subject in need thereof comprising administering a therapeutically effective amount of aripiprazole.

5. The method of claim 5, wherein the polyglutamine disease is selected from the group consisting of SCA type 1, SCA type 2, SCA type 6, SCA type 7, SCA type 17, MJD/SCA3, Huntington's disease, DRPLA, and SBMA.

6. The method of claim 4 or 5, comprising administering aripiprazole in an amount effective to decrease levels of a mutant protein having an expanded polyglutamine tract.

7. The method of any of claims 1-6, wherein the subject has MJD/SCA3.

8. Use of aripirazole in the manufacture of a medicament for use in treatment of MJD/SCA3.

9. Aripiprazole for use in the treatment of MJD/SCA3.

10. The method or use of any of claims 1-9, comprising administering aripiprazole in an amount effective to decrease ataxin-3 levels in an area of the central nervous system of the subject selected from the brainstem, cerebellum, spinal cord, forebrain, and combinations thereof.

11. The method or use of any of claims 1-10, comprising administering aripiprazole in an amount effective to decrease high molecular weight ataxin-3 species.

12. The method or use of any of claims 1-11, comprising administering aripiprazole in an amount effective to decrease ataxin-3 aggregates.

13. The method or use of any of claims 1-12, wherein the subject is a human patient.

14. The method or use of any of claims 1-13, comprising administering aripiprazole orally.

15. The method or use of any of claims 1-13, comprising administering aripiprazole parenterally.

16. The method or use of any of claims 1-15, comprising administering aripiprazole at least once a day.

17. The method or use of any of claims 1-16, comprising administering aripirazole in a dosage of between about 1 mg and about 50 mg.

18. A method of reducing intracellular ataxin-3 levels comprising contacting a cell with an effective amount of aripiprazole.

19. The method of claim 18, wherein the cell is a neuron or a glial cell.

20. The method or use of any of claims 10-19, wherein the ataxin-3 is a mutant ataxin-3 comprising an expanded polyglutamine (polyQ) tract compared to wild-type ataxin-3.
